(19) United States
(12) Patent Application Publication
(10) Pub. No.: US 2019/0290872 A1
(43) Pub. Date: Sep. 26, 2019

(54) TRACHEAL INTUBATION PROCEDURE MONITORING

(71) Applicant: University of Utah Research Foundation, Salt Lake City, UT (US)

(72) Inventors: Sean T. Runnels, Salt Lake City, UT (US); Kai Kuck, Salt Lake City, UT (US); Samer Merchant, Salt Lake City, UT (US)

(21) Appl. No.: 16/289,153

(22) Filed: Feb. 28, 2019

Related U.S. Application Data
(60) Provisional application No. 62/637,152, filed on Mar. 1, 2018.

(51) Int. Cl. A61M 16/04; G16H 40/63

(52) U.S. Cl. CPC A61M 16/0488; A61B 1/267; G16H 40/63

(57) ABSTRACT

A technology is described for monitoring a tracheal intubation procedure. An example method may include receiving procedure data associated with a tracheal intubation procedure from at least one device used to monitor the tracheal intubation procedure. The procedure data received from the at least one device may be analyzed to determine a status of the tracheal intubation procedure and detect irregularities in performing the tracheal intubation procedure. Thereafter, instructions for performing the tracheal intubation procedure which correspond to the status of the tracheal intubation procedure may be provided to an output device, wherein the instructions include information associated with any irregularities detected during performance of the tracheal intubation procedure.

### RELATED APPLICATION

This application is related to U.S. Provisional Application No. 62/637,152 filed on Mar. 1, 2018, which is incorporated herein by reference.

### BACKGROUND

Tracheal intubation is a medical procedure in which an endotracheal tube is placed into the trachea (windpipe) through the mouth or nose. The procedure is frequently performed on critically injured, ill, or anesthetized patients to facilitate ventilation of the lungs, including mechanical ventilation, and to prevent the possibility of asphyxiation or airway obstruction. The vast majority of tracheal intubations involve the use of a viewing instrument of one type or another. One instrument is the laryngoscope, which is used by anesthesiologists, critical care clinicians, and emergency care personnel to facilitate intubating patients in need of mechanical ventilation to protect the patient's airway. The modern conventional laryngoscope comprises a handle, a light, and a set of interchangeable blades, which can be straight or curved.

### SUMMARY

A technology is described for monitoring a tracheal intubation procedure using procedure data generated by one or more devices and executing actions based on the status of the tracheal intubation procedure. In one example, procedure data associated with a tracheal intubation procedure being performed on a patient can be used to monitor the status of the procedure and output information associated with any irregularities detected during the procedure. The information can be used by a healthcare provider to decrease the amount of time to perform the tracheal intubation procedure and avoid or reduce errors that may occur during performance of tracheal intubation procedure. For example, various devices associated with performing a tracheal intubation, including, but not limited to, an intubation device, patient monitoring devices, and/or room monitoring devices, can generate procedure data which can be analyzed by a computing device to determine the status of the tracheal intubation being performed. The status of the tracheal intubation can be evaluated against procedure feedback data used to improve the tracheal intubation procedure. For example, the procedure feedback data can be used to improve the amount of time used to perform a tracheal intubation and avoid or reduce the number of errors that may occur during performance of the tracheal intubation.

Evaluation of the status of the tracheal intubation procedure may identify an action that can be executed that may improve the tracheal intubation procedure by reducing an amount of time to perform a tracheal intubation and/or avoid or reduce errors associated with performing a tracheal intubation. For example, an action may reduce the amount of time to perform the tracheal intubation by outputting instructions for performing the tracheal intubation, output information associated with a number of attempts that have been made to intubate a patient, outputting an amount of time that has elapsed since starting the tracheal intubation, as well as other actions that may improve a time to perform a tracheal intubation. Also, an action may avoid errors associated with performing the tracheal intubation by outputting video that includes visual indicators overlaying a video image of a patient airway used to identify anatomical features of the patient airway and a path for inserting an intubation device to a trachea opening, providing haptic feedback to an intubation device when the intubation device deviates from the path for inserting the intubation device to the trachea opening identified by the visual indicators, outputting an alert indicating that a number of attempts to perform the tracheal intubation meets an attempt threshold, sending an alert message requesting assistance with performing the tracheal intubation procedure in response to a determination that the number of attempts to perform the tracheal intubation meets the attempt threshold, as well as other actions that may avoid or reduce errors associated with performing a tracheal intubation.

After identifying an action that may improve the performance of the tracheal intubation has been identified, the action may be executed to provide feedback to a healthcare provider who is performing the tracheal intubation and/or to healthcare providers who may be assisting in the tracheal intubation procedure. Procedure data generated during the tracheal intubation procedure can be collected and the procedure data can be sent to a remote data store for post-procedure analysis of the tracheal intubation procedure. The procedure data sent to the remote data store can contain information associated with performing the tracheal intubation procedure and any irregularities detected during performance of the tracheal intubation procedure. The post-procedure analysis of the procedure data can be performed to identify actions that may improve a time to perform the tracheal intubation procedure and may reduce a number of errors that can occur during performance of the tracheal intubation procedure.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a block diagram illustrating an example system used to monitor a tracheal intubation to determine a status of the tracheal intubation and execute actions based on the status.

FIG. 2 is a block diagram illustrating components of an example system on which the present technology may be executed.

FIG. 3 is a flow diagram that illustrates an example method for executing an action based on a status of a tracheal intubation procedure.

FIG. 4 is a flow diagram illustrating an example method for monitoring a tracheal intubation procedure.

FIG. 5 illustrates a computing device on which modules of this technology may execute.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “an electrode” includes reference to one or more of such devices and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

The technology described herein relates to systems and methods for monitoring a tracheal intubation procedure. Anesthesiologists, critical care clinicians, and emergency care personnel may perform a tracheal intubation on a patient to protect the patient's airway and provide mechanical ventilation to the patient. Devices, such as laryngoscopes (e.g., configured with cameras, audio devices, and other sensors), patient monitoring devices (e.g., respiratory, neurological, cardiac, etc.), and/or room monitoring devices (cameras, audio devices, environment sensors, etc.) can be used to generate procedure data. For example, an intubation device, such as a laryngoscope, configured with one or more cameras and sensor devices can generate procedure data that includes imaging data for an airway and vocal chords through which an endotracheal tube can be passed, and air composition data for airway gasses flowing through the airway.

Procedure data generated by the devices can be sent to one or more procedure monitoring devices configured to analyze the procedure data to determine a status of the tracheal intubation procedure and detect errors in performing the tracheal intubation procedure. Information associated with the status of the tracheal intubation procedure can be provided to healthcare providers (e.g., via display output, audio output, hepatic output, etc.). For example, a status of a tracheal intubation procedure and irregularities detected during performance of the tracheal intubation procedure can be output to a display or an audio device. Illustratively, status information can include the number of tracheal intubation attempts performed by a healthcare provider, the time that has elapsed since the procedure was started, air composition of the airway (e.g., oxygen and carbon dioxide percentages), physiological information (e.g., location of a stylet, detection of airway abnormalities, etc.), as well as other status information. The status information may assist healthcare providers in performing a tracheal intubation and alert the healthcare providers to any irregularities that may occur during performance of the tracheal intubation.

In one example, procedure data can be collected (e.g., in a remote data store) so that analysis of a tracheal intubation procedure can be performed at a later time. For example, the procedure data can be analyzed to identify actions which may improve a time to perform the tracheal intubation procedure and reduce a number of errors that occur during performance of a tracheal intubation procedure.

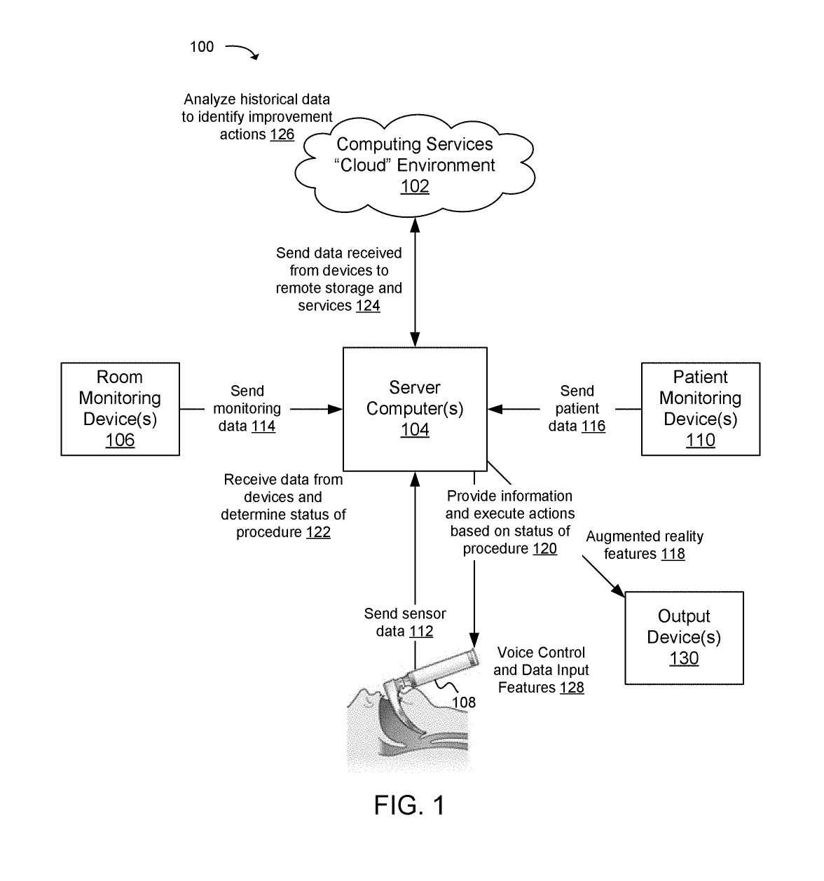

To further describe the present technology, examples are now provided with reference to the figures. FIG. 1 is block diagram that illustrates a high level example of a system 100 used to monitor a tracheal intubation procedure using procedure data provided by sensors and devices and execute actions based on analysis of the procedure data. As illustrated, the system 100 can include a server computer 104 communicatively coupled to an intubation device 108, patient monitoring devices 110, room monitoring device 106, output devices 130, as well as computing services hosted by a computing services “cloud” environment 102.

The devices 106/108/110 may be configured to send procedure data to the server computer 104. Procedure data can include, but is not limited to: sensor data (e.g., image, audio, temperature, humidity, air composition, etc.), patient physiological data (e.g., body temperature, pulse rate, respiration rate, blood pressure, etc.), room monitoring data (e.g., room images, video, audio, motion, etc.), and other data. For example, the intubation device 108 can include sensors configured to send sensor data 112 to the server computer 104, the room monitoring devices 106 may be configured to send monitoring data 114 to the server computer 104, and the patient monitoring devices 110 may be configured to send patient data 116 to the server computer 104.

The server computer 104 may receive procedure data sent by the devices 106/108/110 and analyze the procedure data to determine a status of a tracheal intubation procedure 122. Alternatively, the procedure data can be sent to a computing service hosted in the computing services environment 102 for analysis of the procedure data to determine the status of the tracheal intubation procedure. Illustratively, procedure data can be analyzed to detect that a tracheal intubation attempt has started, detect that a tracheal intubation attempt was successful, and detect how many tracheal intubation attempts were performed. Information related to the status of the tracheal intubation procedure can be provided 120 to healthcare providers via output devices 130 (e.g., display devices, audio devices), as well as an intubation device 108 via haptic feedback.

Based on the status of the tracheal intubation procedure, the server computer 104 may be configured to determine one or more actions that can be taken based on the status of the tracheal intubation procedure. For example, the status of a tracheal intubation procedure may indicate a problem or error in performing a tracheal intubation, such as exceeding a predetermined amount of time allowed to perform the tracheal intubation, exceeding an allowed number of attempts to perform the tracheal intubation, or misplacement of an endotracheal tube in a patient's esophagus.

In one example, a status of a tracheal intubation procedure can be evaluated against procedure feedback data to determine whether an action can be executed to reduce an amount of time to perform the tracheal intubation procedure and/or avoid errors in performing the tracheal intubation procedure. The procedure feedback data can be developed using historical procedure data associated with past tracheal intubations performed on a plurality of patients. For example, historical procedure data for past tracheal intubations can be analyzed to determine how many intubation attempts can be performed without harming a patient and/or how much time can elapse between starting a tracheal intubation procedure and finishing the procedure without harm to a patient. Based on the analysis of the historical procedure data, procedure feedback data can be developed to indicate an attempt threshold for a number of attempts to intubate a patient that can be performed and a time threshold for an amount of time within which to intubate a patient.

In some examples, procedure feedback data can include historical procedure data associated with a patient on who a tracheal intubation procedure is being performed. As an illustration, post-procedure analysis of procedure data can be performed to identify actions to that may reduce a time to perform a tracheal intubation and reduce a number of errors that may occur during performance of the tracheal intubation procedure on a particular patient. As an illustration, a patient may have unique anatomic features that may make tracheal intubation of the patient difficult. Accordingly, procedure feedback data can be developed for the patient and the procedure feedback data can be used to output instructions to an output device 130 (e.g., a video monitor displaying a path for inserting an intubation device 108 into the patient's trachea opening) or to an intubation device 108 (e.g., haptic feedback via an intubation device 108 when the intubation device 108 deviates from a path for inserting the intubation device 108 to the patient's trachea opening).

In response to detecting a problem or error via analysis of procedure data, an action that corresponds to the problem or error detected may be executed 120. As one example, in response to detecting that a predetermined amount of time or information related to a number of attempts allowed to perform the tracheal intubation has been exceeded, a warning can be output to a display or audio device, or via haptic feedback, or a remote alert message requesting assistance may be sent to other healthcare providers. For example, the remote alert message can be a SMS (Short Message Service), an automated phone call, a broadcast message, and the like which is sent to an individual healthcare provider or a group of healthcare providers. Alternatively, a warning light can indicate the number of attempts has reached an intermediate warning stage or a critical concern stage. As another example, in response to detecting a misplacement of an endotracheal tube, an alert can be sent to an output device 130 (e.g., an audio warning), and/or haptic feedback can be provided via an intubation device 108 to indicate to a healthcare provider that the endotracheal tube is misplaced.

Procedure data received from one or more of the devices 106/108/110 can be sent 124 to a remote storage for collection, and services hosted in the computing services environment 102 can be used to analyze 126 historical procedure data in order to identify actions which may improve a tracheal intubation procedure. For example, the historical procedure data can be evaluated to determine whether a time to perform a tracheal intubation can be improved, whether the quality of care in performing the tracheal intubation can be improved, and/or whether a number of errors that occur during performance of a tracheal intubation can be reduced. Procedure feedback data can be developed based on the analysis of the historical procedure data, and actions identified as potentially improving the tracheal intubation procedure can be implemented via the system 100. For example, procedure feedback data, such as attempt thresholds, time thresholds, instructions for performing a tracheal intubation, and the like can be developed, and actions, such as outputting alerts, notifications, instructions, and the like can be executed to potentially reduce the time to perform a tracheal intubation and avoid errors in performing the tracheal intubation.

The system 100 can be configured to provide additional features used to perform a tracheal intubation procedure. In one example, the system 100 can include augmented reality features 118 used to perform a tracheal intubation. As an illustration, augmented reality can be used to overlay visual indicators on a video image of a patient airway. The visual indicators may identify anatomical features and a path for inserting an intubation device 108 to a trachea opening of a patient. The augmented video image can be output to a display located on an intubation device 108, an augmented reality headset, or to another output device 112 capable of displaying the augmented video image.

In another example, the system 100 can include voice control features and/or data input features 128. For example, an intubation device 108 can include an audio device used to detect speech and the speech can be analyzed to detect voice instructions linked to recording functions (e.g., audio/video recording on/off, or audio note recording) which can be used by a healthcare provider to add video and/or audio recordings to a procedure record generated during a tracheal intubation procedure. Also, an intubation device 108 can include other types of data input devices, such as a medical supply scanning device or reader device used to scan medical supplies (e.g., via barcodes or radio frequency identifications (RFIDs)) used during a tracheal intubation procedure. In one example, a camera integrated into an intubation device 108 can be used as a scanner to identify computer readable codes (e.g. bar codes, QR codes, etc.) for identifying packaging, inventory control, patient identification, or the like. In one example, the camera can be used to capture an image and pattern recognition can be used to interpret the image and perform inventory of items recognized within the image. As a non-limiting example, the image can be processed to count a number of disposable endotracheal tubes in an anesthesiologist's drawer, in some cases, without reading a machine readable code.

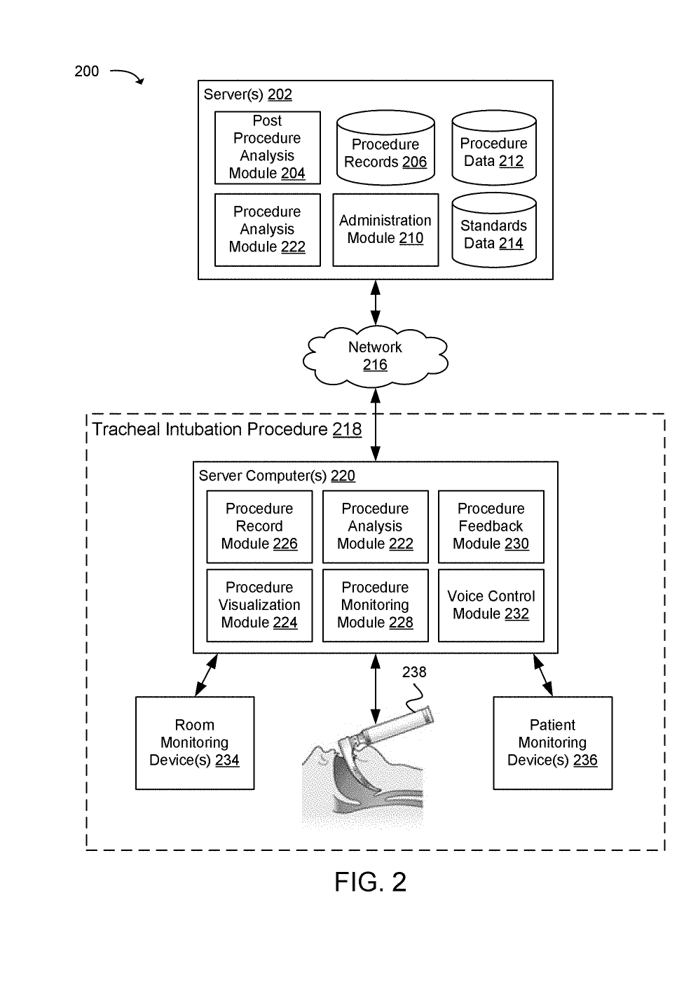

FIG. 2 is a block diagram illustrating example components of a system 200 used to monitor a tracheal intubation procedure 218. The components of the system 200 may include one or more server computers 220 connected to a number of devices 234/236/238 via direct or wireless connections, as well as in network connection with one or more servers 202. The server computer 220 may be configured to monitor a tracheal intubation procedure 218 via procedure data provided by the devices 234/236/238 and provide information associated with the status of the tracheal intubation procedure 218, as well as information associated with any irregularities detected during performance of the tracheal intubation procedure 218.

As illustrated, a server computer 220 can include a procedure monitoring module 228 configured to provide an I/O (Input and Output) interface for procedure data associated with a tracheal intubation procedure 218. One or more devices 234/236/238 may generate procedure data, which may be analyzed to determine a status of the tracheal intubation procedure 218, and procedure information associated with the status of the tracheal intubation procedure 218 can be provided to one or more of the devices 234/236/238.

In one example, the procedure monitoring module 228 may receive procedure data from an intubation device 238. The intubation device 238 may be used to perform a tracheal intubation. The intubation device 238 can include sensors used to generate procedure data and send the procedure data to the procedure monitoring module 228. The sensors included in the intubation device 238 can include: one or more image sensors (cameras), environmental sensors (e.g., oxygen sensor, carbon dioxide sensor, temperature sensor, humidity sensor, etc.), sound sensor (microphone), a medical supply scanning device (e.g., a RFID reader device or bar code scanner device used to track medical supplies), as well as other sensors. In one example, the intubation device 238 can include a set of cameras, where a first camera is positioned in front of a second camera, and images received from the cameras can be stitched together to form an airway image providing an elongated field of view of an airway. For example, a first camera can be positioned near the tip (front end) of a stylet and a second camera can be positioned a distance (e.g., 3-7 cm) behind the first camera so as to provide an elongated view of an airway when the images received from the first camera and the second camera are stitched together.

Also, the procedure monitoring module 228 may receive procedure data from one or more patient monitoring devices 236. The patient monitoring devices 236 may generate procedure data during a tracheal intubation procedure 218 and send the procedure data to the procedure monitoring module 228. Examples of patient monitoring devices 236 can include: respiratory monitoring devices (e.g., pulse oximeter), cardiac monitoring devices, neurological monitoring devices, and other patient monitoring devices.

Moreover, the procedure monitoring module 228 may receive procedure data from one or more room monitoring devices 234. The room monitoring devices 234 can be used to monitor a room environment (e.g., an emergency room, an operating room, a patient's room, etc.) where a tracheal intubation procedure 218 is being performed and send procedure data associated with the room environment to the procedure monitoring module 228. Examples of room monitoring devices 234 can include: cameras (e.g., facial recognition cameras), audio devices (e.g., voice detection and voice recognition devices), environment sensors (e.g., pressure detection sensors, motion detection sensors, LIDAR (Light Detection and Ranging) device, etc.), and other room monitoring devices 234.

In one example, the procedure monitoring module 228 may receive procedure data from the devices 234/236/238 and send the procedure data to a procedure analysis module 222 configured to analyze the procedure data and determine a status of a tracheal intubation procedure 218. The procedure analysis module 222 may be located on the server computer 220, or the procedure analysis module 222 may be hosted on a remote server 202 (e.g., located in a health provider data center or a computing service environment (“cloud” environment)) accessible to the procedure monitoring module 228 via a network 216.

In one example, the procedure analysis module 222 can be used to analyze room environment data generated by the room monitoring devices 234 and airway data generated by the sensors include in the intubation device 238 to detect when a tracheal intubation begins (e.g., via presence of healthcare providers, voice/object/feature recognition, insertion of intubation device 238 into oral cavity, etc.) and when the tracheal intubation ends (e.g., via extraction of the intubation device 238, voice/object/feature recognition, etc.). In another example, the procedure analysis module 222 can be used to analyze airway data (e.g., imaging data, air composition data, and audio data) generated by sensors included in the intubation device 238 to detect a number of attempts to perform a tracheal intubation. In yet another example, the procedure analysis module 222 can be used to analyze patient data generated by a patient monitoring device 236 (e.g., respiratory, cardiac, and/or neurological monitoring device), along with additional procedure data to determine the status of a tracheal intubation procedure 218. After determining a status of the tracheal intubation procedure 218, the procedure analysis module 222 may send status information to the procedure monitoring module 228, and the procedure monitoring module 228 may output the status information to a device 234/236/238 making the status information available to a healthcare provider.

In one example, the procedure analysis module 222 can be used to monitor physiological data to identify airway features, as well as airway abnormalities. For example, physiological data can be monitored to identify airway features which can be used to determine a status of a tracheal intubation procedure 218. For example, a location and/or position of a stylet attached to an intubation device 238 can be determined by identifying airway features within a vicinity of the stylet, and the location and/or position of the stylet can be used in determining the status of the tracheal intubation procedure 218. Physiological data can be monitored to detect an airway abnormality, which can be used to provide feedback to a healthcare provider on how to perform a tracheal intubation procedure 218 in light of the airway abnormality, as well as document the airway abnormality (e.g., for later evaluation and treatment).

Also, the procedure analysis module 222 may be configured to analyze procedure data to detect errors that may occur during performance of a tracheal intubation procedure 218. As one example, the procedure analysis module 222 can be used to analyze imaging data to identify when a stylet attached to the intubation device 238 deviates from a path for inserting the stylet to the trachea opening. As another example, the procedure analysis module 222 can be used to analyze air composition data and/or audio data to identify when an endotracheal tube is inserted into the esophagus instead of the trachea. As yet another example, the procedure analysis module 222 can be used to analyze timer data along with airway data to identify when a tracheal intubation procedure attempt has exceeded a recommended time in which to perform the tracheal intubation procedure 218. In detecting an error in performing a tracheal intubation procedure 218, the procedure analysis module 222 may send error information to the procedure monitoring module 228, and the procedure monitoring module 228 may output the error information to a device 234/236/238 providing a notice of the error to a healthcare provider. As will be appreciated, other examples of using the procedure analysis module 222 to analyze procedure data to determine a status of, and detect errors during, a tracheal intubation procedure, are within the scope of this disclosure.

The server computer 220 may include additional modules that provide functionality related to a tracheal intubation procedure 218. In one example, the server computer 220 may include a procedure visualization module 224 configured to provide visual indicators (via augmented reality) for anatomical features and a path for inserting a stylet to the trachea opening of a patient. The visual indicators can be overlaid on a video image of a patient airway output to a display located on an intubation device 238, an augmented reality headset, or to another device (e.g., patient monitoring device 236). For example, the visual indicators can be used to identify (e.g., label) anatomical features of the patient airway (including airway anomalies) and/or a path for inserting a stylet to the patient's trachea opening. Identification of anatomical features can be performed using one or more techniques, including: laser scanning, color detection, and/or image recognition. In one example, the procedure monitoring module 228 may be used to detect when a tracheal intubation begins (e.g., via procedure data received from the devices 234/236/238) and in response, the procedure monitoring module 228 may instruct the procedure visualization module 224 to output the visual indicators to a display.

The server computer 220 can include a procedure record module 226 configured to create a procedure record 206 for a tracheal intubation procedure 218. The procedure record 206 can include procedure data related to a tracheal intubation procedure 218, such as, billable medical supplies used during the tracheal intubation procedure 218, audio and text data (e.g., procedure notes) related to the tracheal intubation procedure 218, video and image data generated during the tracheal intubation procedure 218, and other procedure data. The procedure record 206 can be created at the time that the tracheal intubation procedure 218 begins and procedure data can be added to the procedure record 206 during and/or after the tracheal intubation procedure 218 ends. For example, the procedure monitoring module 228 may be used to detect when a tracheal intubation procedure 218 begins (e.g., via procedure data received from the devices 234/236/238) and in response, the procedure monitoring module 228 may instruct the procedure record module 226 to create a procedure record 206 for the tracheal intubation procedure 218. As part of creating the procedure record 206, the procedure record module 226 can generate a record identifier for the procedure record 206 and the record identifier can be linked to a patient identifier assigned to a patient who is being intubated. The procedure record module 226 may receive and/or obtain procedure data generated during the tracheal intubation procedure 218 and add the procedure data to the procedure record 206. The procedure record module 226 can save (store) the procedure record 206 to a patient account stored in a remote data store.

The server computer 220 can include a voice control module 232 configured to detect voice instructions linked to recording functions which can be used to record notes during a tracheal intubation procedure 218. In one example, an intubation device 238 can include an audio device used to detect speech and send audio data generated from the detected speech to the voice control module 232. The voice control module 232 can be configured to analyze the voice data for voice instructions (e.g., keywords) linked to functions associated with the voice instructions. As an illustration, a healthcare provider can speak a voice instruction (e.g., “create patient note”, “start recording”, “stop recording”, etc.), which can be received by the audio device included in the intubation device 238 and sent to the voice control module 232. The voice control module 232 can detect the voice instruction and execute a function (e.g., add audio note to procedure record 206, record audio input, stop recording audio input, etc.) associated with the voice instruction.

The server computer 220 can include a procedure feedback module 230 configured to provide healthcare providers with feedback (e.g., audio, visual, and/or haptic) associated with a tracheal intubation procedure 218 by executing an action (e.g., outputting audio, displaying a path for inserting an intubation device to a trachea opening, initiating haptic feedback, the like, or combinations of such actions). In one example, the procedure feedback module 230 can be used to provide intubation procedure feedback based on the status of a tracheal intubation procedure 218. For example, a status of a tracheal intubation procedure 218 can be determined using procedure data received from devices 234/236/238, as described earlier, and the status can be evaluated against procedure feedback data to determine whether to execute an action to reduce a time to perform the tracheal intubation procedure and avoid errors in performing the tracheal intubation procedure. The procedure feedback data can be developed using, for example, historical procedure data associated with past tracheal intubations performed on a plurality of patients. In one example, procedure feedback data can be developed using standards data 214 (e.g., institutional standards of care information) to provide baselines and parameters for evaluating historical procedure data for past tracheal intubation procedures. The procedure feedback data can be linked to one or more actions that can be executed to improve a time to perform a tracheal intubation and avoid errors in performing the tracheal intubation.

As an illustration, procedure data (e.g., an airway image and air composition data) can be analyzed to determine a status of a tracheal intubation procedure. Analysis of the procedure data may show that a stylet is located in a patient's esophagus instead of the patient's trachea. The status may be evaluated against procedure feedback data which indicates that the stylet should be located in the patient's trachea, for example. In response to the evaluation of the status, the procedure feedback module 230 can be used to execute an action linked to the procedure feedback data that provides feedback to a healthcare provider (e.g., via audio output and/or haptic output to an intubation device 238) that the stylet is located in the esophagus. In one example, the procedure feedback module 230 can be used to provide intubation instructions to a healthcare provider, such as step-by-step instructions for inserting an endotracheal tube into a patient's esophagus. The procedure feedback module 230 can interface with the procedure analysis module 222 to detect when an intubation step has been started and completed, allowing the procedure feedback module 230 to guide a healthcare provider through the tracheal intubation procedure 218.

In one example, procedure data 212 for tracheal intubation procedures can be collected at a remote storage location (e.g., a datacenter server 202) and the procedure data 212 (e.g., historical procedure data) can be analyzed using a post procedure analysis module 204 configured to identify actions that may improve a time to perform a tracheal intubation, improve the quality of care in performing the tracheal intubation, and/or reduce a number of errors that occur during performance of the tracheal intubation. In evaluating procedure data 212 for past tracheal intubations, the post procedure analysis module 204 can be configured to use standards data 214 (e.g., institutional standards of care information) to provide baselines and parameters for evaluating the past tracheal intubations. In one example, machine learning can be used to analyze the procedure data 212. Results obtained from analyzing past intubations (e.g., actions identified as improving a tracheal intubation procedure) can be incorporated into procedure feedback data, and the procedure feedback data can be linked to actions that can be executed by the procedure feedback module 230. As an example, results of analyzing past intubations may indicate that performing a tracheal intubation within a specified amount of time is associated with a greater number of successful intubations. This result may be integrated into procedure feedback data for a time threshold to perform a tracheal intubation, and the time threshold may be linked to one or more actions that are performed when the time threshold is met or exceeded. Having developed the procedure feedback data for the time threshold, a status of a tracheal intubation procedure (e.g., an amount of time that has elapsed since starting the procedure) can be evaluated against the time threshold, and if the status indicates that the elapsed time meets or exceeds the threshold, the action linked to the procedure feedback data can be executed (e.g., generating an alert and/or notification). Thus, the system can provide immediate real-time tactical feedback for a single procedure, as well as provide strategic aggregate feedback for future procedures using aggregate information.

The server 202 can include an administration module 210 configured to provide administrative functions associated with billing and inventory management. In one example, the administration module 210 can be used to identify and track medical items used to perform a tracheal intubation and link the medical items to a patient identifier so that the medical items can be billed to a patient associated with the patient identifier. For example, procedure data associated with medical supplies, medical equipment, healthcare providers, and other billable items linked to a tracheal intubation can be sent to the administration module 210, and the administration module 210 can identify the billable items and add the billable items to a patient record. In another example, the administration module 210 can be used to track an inventory of medical supplies used during a tracheal intubation via procedure data sent to the administration module 210. The administration module 210 can interface with an inventory management system (not shown) in order to update inventory records for the medical supplies used during the tracheal intubation.

The various processes and/or other functionality contained within the system 200 may be executed on one or more processors that are in communication with one or more memory modules. The system 200 can include a number of computing devices that are arranged, for example, in one or more server banks or computer banks or other arrangements.

The term “data store” may refer to any device or combination of devices capable of storing, accessing, organizing and/or retrieving data, which may include any combination and number of data servers, relational databases, object oriented databases, cluster storage systems, data storage devices, data warehouses, flat files and data storage configuration in any centralized, distributed, or clustered environment. The storage system components of the data store may include storage systems such as a SAN (Storage Area Network), cloud storage network, volatile or non-volatile RAM, optical media, or hard-drive type media. The data store may be representative of a plurality of data stores as can be appreciated. Thus, the data store and memory device can be located in proximity to the medical environment (e.g. operating room) or remotely located (e.g. separate room, remote building, or network).

API (Application Programming Interface) calls, procedure calls or other network commands that may be made in relation to the modules included in the system 200 may be implemented according to different technologies, including, but not limited to, Representational state transfer (REST) technology or Simple Object Access Protocol (SOAP) technology. REST is an architectural style for distributed hypermedia systems. A RESTful API (which may also be referred to as a RESTful web service) is a web service API implemented using HTTP and REST technology. SOAP is a protocol for exchanging information in the context of Web-based services.

A network 216 may include any useful computing network, including an intranet, the Internet, a local area network, a wide area network, a wireless data network, or any other such network or combination thereof. Components utilized for such a system may depend at least in part upon the type of network and/or environment selected. Communication over the network may be enabled by wired or wireless connections and combinations thereof.

FIG. 2 illustrates that certain processing modules may be discussed in connection with this technology and these processing modules may be implemented as computing services. In one example configuration, a module may be considered a service with one or more processes executing on a server or other computer hardware. Such services may be centrally hosted functionality or a service application that may receive requests and provide output to other services or consumer devices. An API may be provided for each module to enable a second module to send requests to and receive output from the first module. Such APIs may also allow third parties to interface with the module and make requests and receive output from the modules. While FIG. 2 illustrates an example of a system that may implement the techniques above, many other similar or different environments are possible. The example environments discussed and illustrated above are merely representative and not limiting.

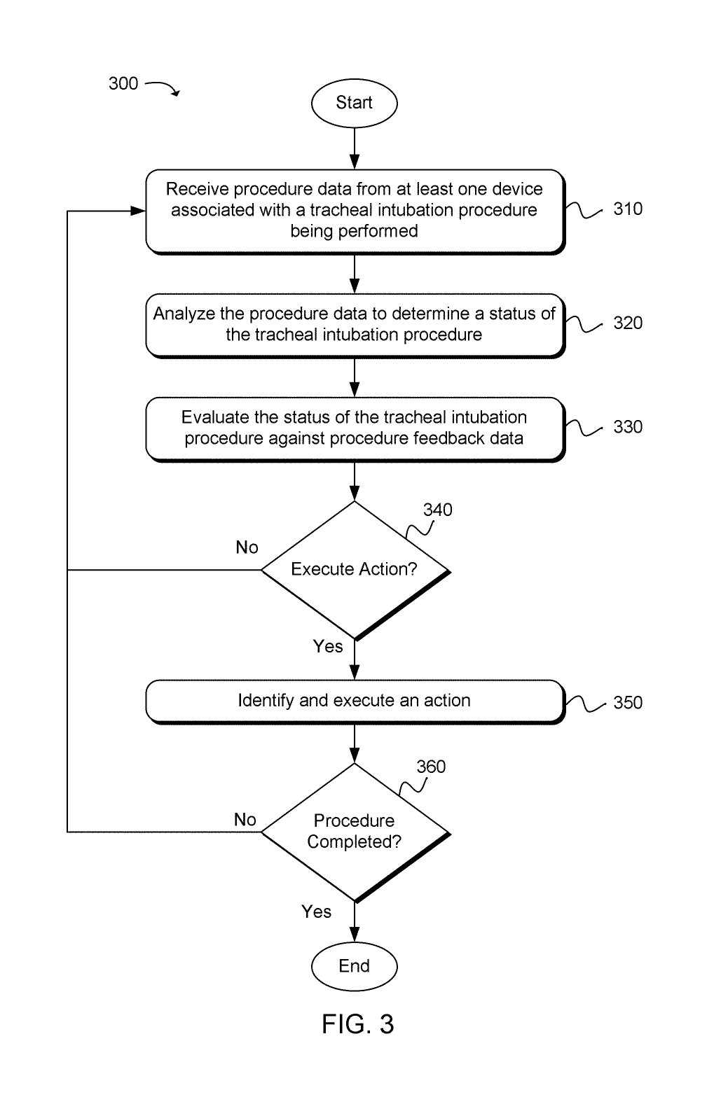

FIG. 3 is a flow diagram illustrating an example method 300 for executing an action based on a status of a tracheal intubation procedure. As described earlier, a tracheal intubation may comprise inserting an endotracheal tube into the trachea of a patient using an intubation device that includes a set of sensors which generate procedure data. Also, patient monitoring devices and/or room monitoring devices can provide procedure data during the performance of a tracheal intubation which can be used to determine a status of the tracheal intubation. The method 300 may include, as in block 310, receiving procedure data from at least one device associated with the tracheal intubation procedure being performed on a patient. Devices that generate the procedure data can include, but are not limited to, an intubation device, patient monitoring devices, and/or room monitoring devices.

As in block 320, the procedure data received from the device(s) can be analyzed to determine a status of the tracheal intubation procedure. For example, procedure data can include sensor data (e.g., image, audio, temperature, humidity, air composition, etc.), patient physiological data (e.g., body temperature, pulse rate, respiration rate, blood pressure, etc.), room monitoring data (e.g., room images, video, audio, motion, etc.), and other data, which can be analyzed to determine that status of the tracheal intubation procedure. The status can be associated with a point of time or step beginning from the start of the tracheal intubation procedure to the end of the tracheal intubation procedure, and the status may indicate one or more conditions that are present associated with a particular point or step in the tracheal intubation procedure. As an illustration, a status may indicate a location of a stylus of an intubation device in a patient's throat, a number of attempts that have been made to intubate the patient, an amount of time that has elapsed since beginning the tracheal intubation procedure, etc.

As in block 330, the status of the tracheal intubation procedure can be evaluated against procedure feedback data. As described earlier, procedure feedback data can be developed using historical procedure data associated with past tracheal intubations performed on a plurality of patients. The status of the tracheal intubation procedure can be evaluated by associating the status with corresponding procedure feedback data to determine whether an action can be executed to improve performance of the tracheal intubation procedure. As one example, a status indicating a location of a stylus in a patient's throat may be evaluated against corresponding procedure feedback data representing the location of the stylus in the patient's throat, which may be linked to one or more actions. As another example, a status indication an amount of time that has elapsed since starting a procedure can be evaluated against corresponding procedure feedback data representing a time threshold for performing the procedure, which may be linked to one or more actions.

Based on the evaluation of the status of the tracheal intubation procedure against the procedure feedback data, as in block 340, a determination whether to execute an action linked to the procedure feedback data can made, which may reduce a time to perform the tracheal intubation procedure and/or avoid errors in performing the tracheal intubation procedure. For example, in the case that the status of the tracheal intubation procedure corresponds to procedure feedback data linked to one or more actions, the actions, as in block 350, can be identified and executed. As one example, procedure feedback data representing the location of the stylus in the patient's throat can be linked to actions for displaying a path for inserting an intubation device to a trachea opening on a display device, outputting audio instructions for inserting the intubation device into the trachea opening, and/or outputting an audio alert to indicate that the stylus is the patient's esophagus instead of the patient's trachea.

As in block 360, in the case that the tracheal intubation procedure is not complete (e.g., an endotracheal tube has not yet been placed into the patient's trachea), the method 300 may continue to analyze procedure data received from the device(s) and evaluate the status of the tracheal intubation procedure to determine whether to execute various actions to reduce the time to perform the tracheal intubation procedure and/or avoid errors in performing the tracheal intubation procedure. In the case that the tracheal intubation procedure is complete, which can be detected via analysis of the procedure data (e.g., procedure data indicating the removal of a stylus from a trachea combined with procedure data indicating airflow through an inserted endotracheal tube), the method 300 may end, and in some examples, the procedure data containing information associated with performing the tracheal intubation procedure and any irregularities detected during performance of the tracheal intubation procedure can be sent to a remote data store for post-procedure analysis of the tracheal intubation procedure.

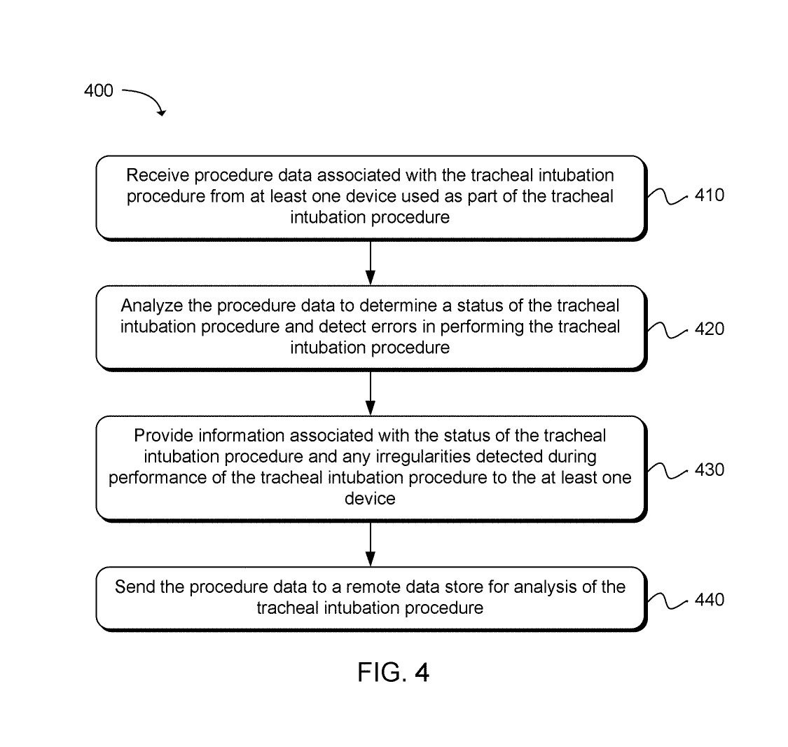

FIG. 4 is a flow diagram that illustrates an example method 400 for monitoring a tracheal intubation procedure. The method 400 may include, as in block 410, receiving the procedure data associated with the tracheal intubation procedure from at least one of these devices used as part of the tracheal intubation procedure. For example, the intubation device used to perform the tracheal intubation can include sensors configured to generate airway data. The airway data generated by the sensors can include: imaging data, air composition data, and audio data. Imaging data can include color images, black and white images, thermal infrared images, tissue spectroscopy data, and the like. Also, the sensors included in the intubation device can include a first camera and a second camera, where the first camera is positioned in front of the second camera. Images received from the first camera and the second camera can be stitched together to form an airway image providing an elongated field of view of an airway, which can be output to a display. As mentioned above, other device can provide procedure data, including a patient monitoring device that generates patient data, and/or a room monitoring device that generates room monitoring data.

As in block 420, the procedure data received from the one or more device can be analyzed to determine a status of the tracheal intubation procedure and detect errors in performing the tracheal intubation procedure, and as in block 430, information associated with the status of the tracheal intubation procedure and any irregularities detected during performance of the tracheal intubation procedure to the at least one device. The status of the tracheal intubation procedure may be linked to an action that provides status information and/or intubation instructions to a healthcare worker via an output device (e.g., a display device, audio device, haptic feedback device, etc.). Determining a status linked to an action may result in executing the action.

As one example, determining a status of a trachea intubation can include analyzing airway data to detect a number of attempts to perform a tracheal intubation, and an action linked to the status can include outputting the number of attempts to perform the tracheal intubation to a display or an audio device.

As another example, determining a status of a trachea intubation can include determining that the number of attempts to perform the tracheal intubation meets an attempt threshold, and an action linked to the status can include sending an alert message requesting assistance in performing the tracheal intubation.

As yet another example, determining a status of a trachea intubation can include detecting a first attempt to perform the tracheal intubation (via analysis of procedure data provided by one or more devices), and an action linked to the status can include starting a timer used to track an amount of time that has elapsed since starting the tracheal intubation. The status of the timer can be monitored in order to determine whether a time threshold to perform the tracheal intubation has been exceeded, and in the case that the timer exceeds the time threshold, an action linked to the status of timer can include sending an alert message requesting assistance with the tracheal intubation.

As in block 440, procedure data received from the one or more devices can be sent to a remote data store for analysis of the tracheal intubation procedure. The remote data store may be used to store historical procedure data. In one example, the historical procedure data may be analyzed using machine learning or by a healthcare provider to identify actions that may improve a time to perform the tracheal intubation procedure, improve a quality of care in performing the tracheal intubation procedure, and/or reduce a number of errors that occur during performance of the tracheal intubation procedure. Actions identified as improving the tracheal intubation procedure may be included in instructions for performing tracheal intubations.

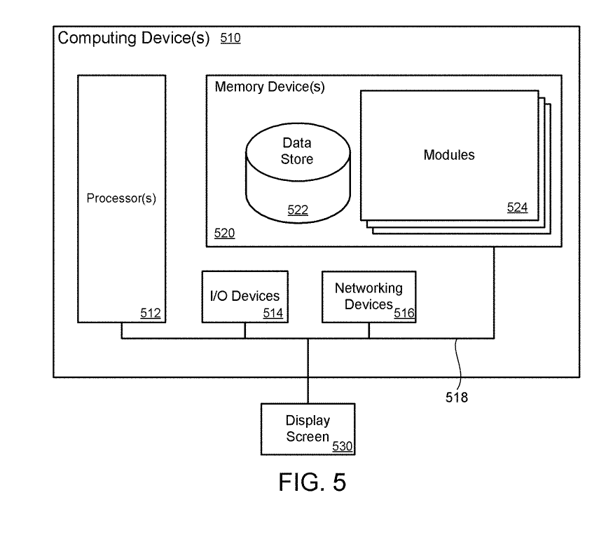

FIG. 5 illustrates a computing device 510 on which modules of this technology may execute. The computing device 510 may include one or more processors 512 that are in communication with memory devices 520. The computing device 510 may include a local communication interface 518 for the components in the computing device. For example, the local communication interface 518 may be a local data bus and/or any related address or control busses as may be desired.

The memory device 520 can contain modules 524 that are executable by the processor(s) 512 and data for the modules 524. For example, the memory device 520 may include a procedure monitoring module, procedure analysis module, procedure feedback module, procedure record module, procedure visualization module, and other modules. The modules 524 may execute the functions described earlier. A data store 522 may also be located in the memory device 520 for storing data related to the modules 524 and other applications along with an operating system that is executable by the processor(s) 512.

Other applications may also be stored in the memory device 520 and may be executable by the processor(s) 512. Components or modules discussed in this description that may be implemented in the form of software using high programming level languages that are compiled, interpreted or executed using a hybrid of the methods.

The computing device may also have access to I/O (input/output) devices 514 that are usable by the computing devices. One such I/O device can include a display screen 530. Networking devices 516 and similar communication devices may be included in the computing device. The networking devices 516 may be wired or wireless networking devices that connect to the Internet, a LAN, WAN, or other computing network.

The components or modules that are shown as being stored in the memory device 520 may be executed by the processor(s) 512. The term “executable” may mean a program file that is in a form that may be executed by a processor 512. For example, a program in a higher level language may be compiled into machine code in a format that may be loaded into a random access portion of the memory device 520 and executed by the processor 512, or source code may be loaded by another executable program and interpreted to generate instructions in a random access portion of the memory to be executed by a processor. The executable program may be stored in any portion or component of the memory device 520. For example, the memory device 520 may be random access memory (RAM), read only memory (ROM), flash memory, a solid state drive, memory card, a hard drive, optical disk, floppy disk, magnetic tape, or any other memory components.

The processor 512 may represent multiple processors and the memory 520 may represent multiple memory units that operate in parallel to the processing circuits. This may provide parallel processing channels for the processes and data in the system. The local interface 518 may be used as a network to facilitate communication between any of the multiple processors and multiple memories. The local interface 518 may use additional systems designed for coordinating communication such as load balancing, bulk data transfer and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, non-transitory media such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A system for monitoring a tracheal intubation procedure, comprising:
at least one processor;
a memory device including instructions that, when executed by the at least one processor, cause the system to:
receive procedure data from at least one device associated with the tracheal intubation procedure being performed;
analyze the procedure data to determine a status of the tracheal intubation procedure;
evaluate the status of the tracheal intubation procedure against procedure feedback data to determine whether to execute an action to reduce a time to perform the tracheal intubation procedure and avoid errors in performing the tracheal intubation procedure, wherein the procedure feedback data is developed using historical procedure data associated with past tracheal intubations performed on a plurality of patients, wherein the procedure feedback data optionally includes historical procedure data associated with a patient on who the tracheal intubation procedure is being performed;
identify the action based at least in part on the evaluation of the status of the tracheal intubation procedure against the procedure feedback data;
execute the action to reduce the time to perform the tracheal intubation procedure and avoid errors in performing the tracheal intubation procedure; and
send the procedure data containing information associated with performing the tracheal intubation procedure and any irregularities detected during performance of the tracheal intubation procedure to a remote data store for post-procedure analysis of the tracheal intubation procedure.

2. The system in claim 1 wherein the at least one device includes an intubation device used to perform a tracheal intubation that includes one or more sensors configured to generate the procedure data, which includes airway data.

3. The system in claim 2, wherein the airway data generated by the intubation device includes one or more of: imaging data, air composition data, and audio data.

4. The system in claim 2, wherein the status of the tracheal intubation procedure includes a number of attempts to perform a tracheal intubation detected via analysis of the airway data, and the procedure feedback data includes an attempt threshold for performing the tracheal intubation.

5. The system in claim 4, wherein the action includes one or more of:
outputting information related to the number of attempts to perform the tracheal intubation;
outputting an alert indicating that the number of attempts to perform the tracheal intubation meets an attempt threshold; and
sending an alert message requesting assistance with performing the tracheal intubation procedure in response to a determination that the number of attempts to perform the tracheal intubation meets the attempt threshold.

6. The system in claim 4, wherein the status of the tracheal intubation procedure includes an amount of time that has elapsed since initiating a tracheal intubation detected via analysis of the airway data, and the procedure feedback data includes a time threshold for performing the tracheal intubation.

7. The system in claim 6, wherein the action includes one or more of:
outputting information related to the amount of time that has elapsed since initiating the tracheal intubation;
outputting an alert indicating that the time threshold to perform the tracheal intubation has been exceeded; and
sending an alert message requesting assistance with performing the tracheal intubation procedure in response to a determination that the time threshold to perform the tracheal intubation has been exceeded.

8. The system in claim 1, wherein the post-procedure analysis of the procedure data is performed to identify actions to improve a time to perform the tracheal intubation procedure and to reduce a number of errors that occur during performance of the tracheal intubation procedure.

9. A computer implemented method for monitoring a tracheal intubation procedure, comprising:
receiving procedure data associated with the tracheal intubation procedure from at least one device used to monitor the tracheal intubation procedure;
analyzing the procedure data to determine a status of the tracheal intubation procedure and detect irregularities in performing the tracheal intubation procedure; and
outputting instructions for performing the tracheal intubation procedure to an output device, wherein the instructions correspond to the status of the tracheal intubation procedure, and the instructions include information associated with the irregularities detected during performance of the tracheal intubation procedure.

10. The method of claim 9, wherein the at least one device used to monitor the tracheal intubation procedure includes: an intubation device configured with a set of sensors that generate the procedure data, a patient monitoring device that generates the procedure data, or a room monitoring device that generates the procedure data.

11. The method of claim 9, wherein outputting the instructions for performing the tracheal intubation procedure further comprise outputting audio to an audio device containing the instructions that correspond to the status of the tracheal intubation procedure and the information associated with any irregularities detected during performance of the tracheal intubation procedure.

12. The method of claim 9, wherein the at least one device includes an intubation device that has a first camera and a second camera, wherein the first camera is positioned in front of the second camera, and images received from the first camera and the second camera are stitched together to form an airway image providing an elongated field of view of an airway and the airway image is output to a display device.

13. The method of claim 9, wherein outputting the instructions for performing the tracheal intubation procedure further comprise outputting video to a display device to include visual indicators that overlay a video image of a patient airway, wherein the visual indicators identify anatomical features of the patient airway and a path for inserting an intubation device to a trachea opening.

14. The method of claim 13, further comprising providing haptic feedback to the intubation device when the intubation device deviates from the path for inserting the intubation device to the trachea opening identified by the visual indicators.

15. The method of claim 9, further comprising:
sending the procedure data to a remote data store used to collect historical procedure data associated with a plurality of tracheal intubation procedures; and
analyzing the historical procedure data, using machine learning and/or by a healthcare provider, to identify actions that improve a time to perform the tracheal intubation procedure, improve a quality of care in performing the tracheal intubation procedure, reduce a number of errors that occur during performance of the tracheal intubation procedure, and/or develop quality of care standards.

16. The method of claim 15, wherein the instructions for performing the tracheal intubation procedure are based at least in part on the actions identified as improving the tracheal intubation procedure.

17. The method of claim 15, wherein the procedure data includes physiological data including current patient physiological data.

18. A non-transitory machine readable storage medium having instructions embodied thereon, the instructions when executed by a processor:
receive procedure data from an intubation device being used to perform a tracheal intubation procedure, wherein the intubation device includes at least one sensor used to generate the procedure data;
analyze the procedure data to determine a status of the tracheal intubation procedure and detect errors in performing the tracheal intubation procedure;
provide information associated with the status of the tracheal intubation procedure and any irregularities detected during performance of the tracheal intubation procedure to an output device; and
send the procedure data to a remote data store.

19. The non-transitory machine readable storage medium in claim 18, wherein the at least one sensor included in the intubation device is data reader configured to detect formatted data containing an identifier for at least one of: a patient, healthcare provider, location, or medical item.

20. The non-transitory machine readable storage medium in claim 18, wherein the instructions that when executed by the processor further:
create at least a portion of a procedure record for the tracheal intubation procedure, wherein a record identifier generated for the procedure record is linked to a patient identifier;
add the procedure data to the procedure record, wherein the procedure data includes identifiers for billable medical supplies used during the tracheal intubation procedure, audio data related to the tracheal intubation procedure, text data related to the tracheal intubation procedure, imaging data generated during the tracheal intubation procedure, or image data generated during the tracheal intubation procedure; and
save the procedure record to a patient account stored in the remote data store.

21. The non-transitory machine readable storage medium in claim 20, wherein the instructions that when executed by the processor further send the procedure data to an inventory management system used to track medical items used to perform the tracheal intubation procedure.
